(19) United States
(12) Patent Application Publication
(10) Pub. No.: US 2020/0353215 A1
(43) Pub. Date: Nov. 12, 2020

(54) System and Methods for Left Atrial Access

(71) Applicant: Baylis Medical Company Inc., Montreal (CA)

(72) Inventors: Gareth Davies, Toronto (CA); Eduardo Moriyama, Richmond (CA)

(21) Appl. No.: 16/867,132

(22) Filed: May 5, 2020

Related U.S. Application Data
(60) Provisional application No. 62/843,883, filed on May 6, 2019.

(51) Int. Cl. A61M 25/01

(52) U.S. Cl. CPC A61M 25/0133; A61M 2025/0681

(57) ABSTRACT

A method and apparatus are disclosed for puncturing tissue within a patient, comprising accessing a hepatic vein within the patient using an access device and inserting a flexible puncture device into the hepatic vein. The flexible puncture device advances from the hepatic vein to an inferior vena cava, and to the patient's heart. The next step is advancing a supporting catheter comprising a lumen over the flexible puncture device, whereby the flexible puncture device is positioned within the lumen and supported by the supporting catheter. The distal tip of the flexible puncture device and supporting catheter are positioned against a target tissue site and creating a puncture in said tissue, and the flexible puncture device is advanced through the puncture in the tissue.

### TECHNICAL FIELD

The disclosure relates generally to medical methods for gaining access to various tissue sites. More particularly, the disclosure relates to a system and methods for accessing the left side of a heart.

### BACKGROUND OF THE ART

It is often required to create a perforation in the atrial septum to gain access to the left side of the heart interventionally to study or treat electrical or morphological abnormalities. Gaining access in this way is sometimes known as a transseptal procedure. In order to carry out a transseptal procedure, it is necessary to gain access to the right side of the heart beforehand. The heart may be accessed from a superior approach (i.e., by gaining access from an access point above the heart, for example from the jugular vein through the superior vena cava), or alternatively access may be obtained from the inferior approach (by gaining access to the heart from an access point below the heart, for example from the femoral vein through the inferior vena cava). Once there is access to the right atrium, a puncture device is utilized in order to puncture through tissue, for example across a septum of the heart to gain access from the right atrium into the left atrium of the heart.

### BRIEF DESCRIPTION OF THE DRAWINGS

In order that the invention may be readily understood, embodiments of the invention are illustrated by way of examples in the accompanying drawings, in which:

FIG. 1 is an illustration of the major blood vessels of the liver;

FIGS. 2A-2B illustrate a system used in an embodiment of the invention;

FIGS. 3A-3G illustrate the steps of performing a transseptal procedure, in accordance with an embodiment of the present invention; and

FIG. 4 is a flow diagram showing a method of performing a transseptal procedure, in accordance with an embodiment of the present invention.

### DETAILED DESCRIPTION

In accordance with an embodiment of the present invention, a method is disclosed for performing a transseptal puncture procedure using a flexible RF device and steerable sheath, the method comprising:

In one broad aspect, embodiments of the present invention comprise a method and apparatus for puncturing tissue within a patient, comprising:
(a) accessing a hepatic vein within the patient using an access device;
(b) inserting a flexible puncture device into the hepatic vein;
(c) advancing the flexible puncture device from the hepatic vein to an inferior vena cava of the patient;
(d) advancing the flexible puncture device through the inferior vena cava to a heart of the patient;
(e) advancing a supporting catheter comprising a lumen over the flexible puncture device, whereby the flexible puncture device is within the lumen and supported by the supporting catheter;
(f) positioning a distal tip of the flexible puncture device and supporting catheter against the target tissue site and creating a puncture in said tissue; and
(g) advancing the flexible puncture device through the target tissue.

In some embodiments, the supporting catheter supports the flexible puncture device to enable force and torque to be transmitted to a distal end of the supporting catheter and puncture device.

In some embodiments, the flexible puncture device is energy based.

In some embodiments, the flexible puncture device is a radiofrequency wire.

In some embodiments, the flexible puncture device is a pigtail wire.

In some embodiments, the flexible puncture device is a J-tip wire.

In some embodiments, the flexible puncture device is sufficiently flexible to be tracked through vasculature of the patient.

In some embodiments, the supporting catheter is a steerable sheath.

In some embodiments, after step (g), the method further comprising:
(h) anchoring the flexible puncture device after puncturing through the target tissue site, to maintain access through the target tissue site to the other side of the target tissue site, in order to allow one or more additional devices to be tracked over the flexible puncture device to the other side of the target tissue site.

In some embodiments, after step (h), the method further comprising:
(i) performing a left heart procedure.

In some embodiments, after step (i), the method further comprising:
(j) removing the devices and closing the hepatic access.

In some embodiments, the target tissue is a septum of the heart.

In some embodiments, step (f) comprises dropping the device down from a superior vena cava into the heart of the patient to locate a fossa along the septum of the heart to position the flexible puncture device at the fossa.

In some embodiments, the step of position the device at the target tissue site comprises actuating the steerable sheath to position the flexible puncture device at a fossa along a septum of the heart.

In some embodiments, the supporting catheter comprises a dilator and a sheath, wherein step (c) comprises inserting the dilator over the flexible puncture device and inserting the sheath over the dilator.

In some embodiments, the sheath is a steerable sheath, and the dilator is substantially flexible to conform to the curve of the steerable sheath that is achieved through actuation of the steerable sheath.

In some embodiments, after step (g), the method further comprising:
(h) advancing the dilator through the puncture in the tissue over the flexible puncture device.

In some embodiments, after step (h), the method further comprising:
(i) advancing the sheath through the puncture in the tissue over the dilator.

In some embodiments, after step (g), the method further comprising:
(h) advancing the sheath through the puncture in the tissue over the flexible puncture device

In some embodiments, the hepatic vein is a right hepatic vein.

In some embodiments, the hepatic vein is a middle or left hepatic vein.

In some embodiments, the hepatic vein is a lower hepatic vein.

In some embodiments, the access device is a Tuohy needle.

In some embodiments, prior to step (a), the method further comprises visualizing hepatic veins in the patient and selecting a hepatic vein.

In some embodiments the flexible puncture device comprises a lumen and one or more apertures.

In some medical procedures, it may be desirable to reach a target tissue site within a patient's body in order, for example, to provide access for transseptal punctures. In order to initially reach the target tissue site, access may be provided into and/or through vasculature using a guidewire. A sheath and/or dilator assembly may then be advanced over the guidewire, and the sheath may be used to guide the dilator, as well as any other devices positioned through the assembly, to the desired target tissue site.

In some such procedures, a particular entry point into the patient's vasculature may be dictated by treatment requirements, anatomical considerations, or other factors. For example, patients with occluded or stenosed vasculature may require an alternate entry point. Occasionally, due to abnormalities of the venous system such as azygous continuation of the inferior vena cava or thrombosis or obliteration of the iliofemoral veins, it may not be possible to gain access to the right atrium using a femoral approach.

In other such procedures, “traditional” entry sites (such as femoral venous entry or jugular vein entry) are used to provide access to auxiliary devices for the procedure. Examples of auxiliary devices include intracardiac echocardiography catheters and coronary sinus catheters. Additionally, alternate methods to access the heart for cardiac procedures may be used to preserve femoral and jugular access for future intervention/treatment as multiple interventions may result in occlusions of major venous accesses.

As mentioned above, in certain procedures, a particular tissue puncture site may be required while the entry points into the vasculature or the vasculature pathways may be somewhat restricted. In such cases, delivering treatment apparatus from the entry point to the tissue puncture site is difficult and/or may require many device exchanges. This may be due to, for example, the curvature and/or tortuosity of the vasculature within that region of the body.

Puncturing certain tissue sites while being limited to particular entry points also often requires exchanging devices (such as puncture devices, guidewires, sheaths, and dilators) multiple times, with each device performing a specific function during the course of the procedure. For example, current methods of accessing a heart chamber on the left side of the heart using a transhepatic access approach use a needle in order to carry out a transseptal puncture. This type of method typically involves multiple exchanges of the needle, guidewire, sheaths, and dilators.

Certain limitations may be associated with the use of needle or other rigid devices for carrying out a transseptal puncture procedure. These limitations may include one or more of:

(1) need for a separate exchange or guidewire to gain access to the SVC resulting in multiple device exchanges on the right side;

(2) the use of a needle may require multiple device exchanges in order to complete the procedure;

(3) difficulty in correcting placement of the puncture device after insertion within the right atrium if the target location on the fossa is missed;

(4) lack of repeatability for certain aspects of the procedure for completing the puncture in an effective and timely manner;

(5) the puncture device may not provide sufficient atraumacity and may result in excessive force being applied to puncture tissue resulting in damage to tissue;

(6) possible risk of trauma to the structures within the left atrium following puncture due to the force of advancement;

(7) lack of adequate anchoring after puncture to maintain access;

(8) need for an additional exchange on the left side requiring removal of the puncture device and advancement of another wire (such as a pigtail wire) to facilitate anchoring; and/or

(9) trackability to allow additional devices to be tracked over the wire once in the left side.

The system of the present invention provides several advantages corresponding to the aforementioned limitations where providing a substantially flexible atraumatic puncture device such as an RF wire in combination with a supporting catheter that is selectively usable with the substantially flexible atraumatic puncture device, provides at least the following advantages:

a) The system enables the supporting catheter to be advanced over the RF wire allowing the RF wire to simultaneously function as an exchange or guidewire. A guidewire is flexible which provides the ability to navigate difficult anatomy while being advanced through a body lumen. Guidewires are typically atraumatic and are therefore less damaging to the body lumen as it is being manipulated while traversing the lumen. Because needles are typically rigid and comprise a sharp tip, they are not suitable for use as a guidewire. An RF wire of the present system, on the other hand, comprises an atraumatic tip and is flexible—these features allow it to better function as an exchange or guidewire, thereby eliminating the need to exchange the puncturing device for a guidewire, which would be required if a needle is used as the puncturing device. Reducing the number of exchanges needed helps to streamline work flow on the right side of the heart which in turn may reduce procedural time and complexity,

b) The system enables repeatability of the drop-down procedure by enabling partial removal or partial retraction or withdrawal of the supporting catheter to enable re-positioning and/or re-advancement of the RF puncturing device such as the RF wire within the superior vena cava (SVC) without requiring an additional exchange.

c) The system additionally enables removal of the supporting catheters after puncture while leaving the RF wire positioned in the left atrium: (i) to enable anchoring within the left side, without requiring an additional exchange, thereby enhancing procedural safety or efficiency; and/or (ii) to allow the RF wire to be used as a guidewire to subsequently allow other devices to be tracked over the wire. The benefit of minimizing exchanges, in addition to reducing procedure time and the number of steps required, is minimizing risk of infection. This is particularly important on the left side of the heart, where any unnecessary exchanges may lead to increased risks, such as risk of embolism or strokes.

The present inventors have conceived and reduced to practice novel and unique methods to facilitate efficient and repeatable puncture of a target tissue while allowing vascular access from the hepatic vein a patient's body. In some embodiments, the target tissue is the septum of the heart.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As an overview of the present invention, some embodiments of the method utilize a two-part assembly comprising a flexible puncture device and a support catheter such as a sheath. The flexible puncture device (e.g., an RF wire) is inserted in the hepatic vein. Because the RF wire is flexible and atraumatic, it may be used as an exchange or guidewire. This eliminates the need for a separate guide/exchange wire to be used for initial access into the superior vena cava (SVC). The sheath can then be used selectively—the sheath can be advanced into the SVC to provide adequate force transmission to facilitate the drop-down procedure to locate the fossa. If the initial pass at locating the fossa is unsuccessful, the two-part assembly enables partial removal or withdrawal of the sheath to enable the RF wire to be repositioned. The sheath may then be re-advanced or re-positioned to provide adequate stiffness and force transmission to repeat the drop-down procedure to locate the fossa and to provide adequate support to facilitate puncture using the RF wire and to facilitate crossing with the RF wire. In other embodiments, a steerable sheath can be used to aim and guide the RF wire to locate the fossa. As such, the RF wire and sheath work together to puncture using the RF wire and cross into the left side after the puncture is complete. The sheath may be removed thereafter leaving the flexible RF wire within the left side of the heart. Thus, the flexible RF wire is usable independently from the sheath to facilitate anchoring, facilitate tracking, to minimize left side exchanges, to minimize risk of embolisms, and to minimize the risk of trauma. In another embodiment, after crossing into the left side of the heart, the RF wire is removed leaving the sheath within the left side of the heart. The sheath can then be used to facilitate the left heart procedure.

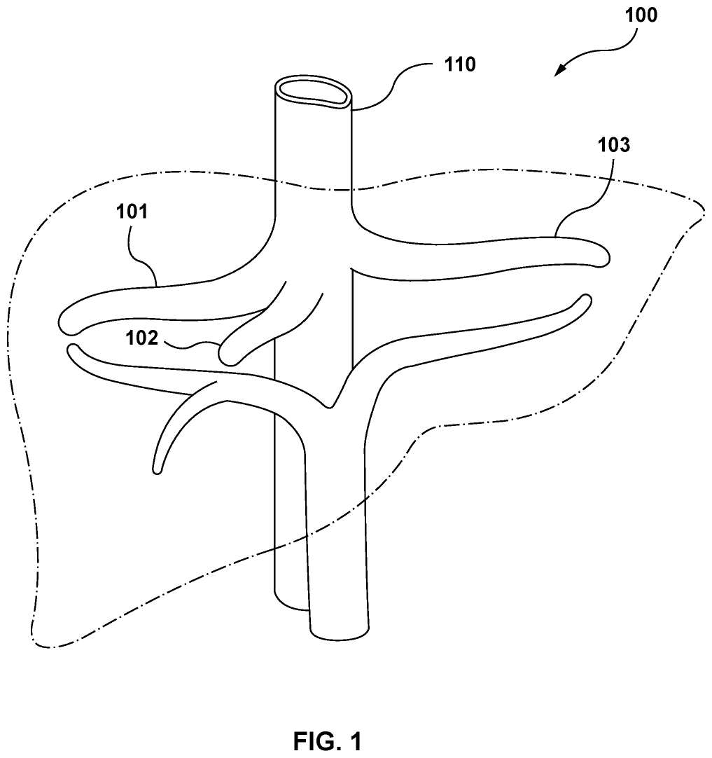

FIG. 1 illustrates, in dotted outline, the liver 100, and further illustrates the vascular anatomy of the liver. More specifically, the inferior vena cava (IVC) 110 is shown. IVC 110 branches to the right hepatic vein 101, the middle hepatic vein 102, and the left hepatic vein 103. In accordance with the current invention, entry to the IVC is gained through a hepatic vein. In an embodiment, the right hepatic vein 101 is used to access the IVC. In other embodiments, the middle hepatic vein 102 or left hepatic vein 103 is used to access the IVC. In other embodiments of the invention, one of the lower hepatic veins (not shown) is used to access the IVC. Ultrasound or other imaging techniques may be used to visualize the hepatic veins to facilitate access to the hepatic vein. In some embodiments of the invention, access to the hepatic vein is provided by an access device such as a needle (such as a Tuohy needle not shown). In some embodiments, the needle is 20 gauge or larger. In a specific embodiment, a 17 gauge needle is used to gain entry to a hepatic vein.

In accordance with some embodiments of the present invention, details of the devices are disclosed in application number PCT/IB2013/060287 and publication number WO2015019132, in application number PCT/IB2017/056777 and publication number WO2018083599, and in application number PCT/IB2017/050065 and publication number WO2017118948A1 which are incorporated herein by reference in their entirety.

In an embodiment, an assembly is provided for puncturing tissue, where the assembly comprises a flexible puncturing device for puncturing tissue via delivery of energy. The puncturing device preferably comprises an atraumatic tip to allow the puncturing device to also be used as an exchange or guidewire. The assembly additionally comprises supporting catheters, such as a sheath and/or dilator, for supporting the flexible puncturing device. The supporting catheters are operable to be selectively usable with the substantially flexible puncturing device and is detachable or removable therefrom. In some embodiments, the flexible puncturing device is an energy based device for delivering energy to the tip of the puncturing device in order to puncture the septum. In some embodiments, the flexible puncturing device has a lumen and at least one aperture. The lumen and aperture may combine to form a pressure transmitting lumen. The flexible puncturing device may be operable to be coupled to a pressure sensing mechanism, such as a pressure sensor, to measure the pressure transmitted through the lumen. In some embodiments, fluid (such as imaging or contrast fluid) is injected through the lumen and at least one aperture.

The assembly enables the flexible energy based puncturing device to be usable independently from the supporting catheters as an exchange or guidewire during a portion of the procedure and to be usable in co-operation with other devices for puncturing tissue during another portion of the procedure. This reduces the number of exchanges needed by allowing the flexible energy based puncture device to be used for puncturing tissue, and as an exchange wire. The decoupling of the puncturing device of the assembly from the supporting catheters, additionally enables the repositioning of the puncture device. For example, supporting catheters can be removed if the flexible puncturing device is not positioned at the desired target location. Once removed, the flexible puncturing device can be repositioned to a desired location after which the supporting catheters may be re-advanced over the flexible puncturing device. The supporting catheters facilitate positioning of the energy delivery portion of the flexible puncturing device against the desired target tissue location and may additionally reduce procedure complexity and enhance procedural efficiency.

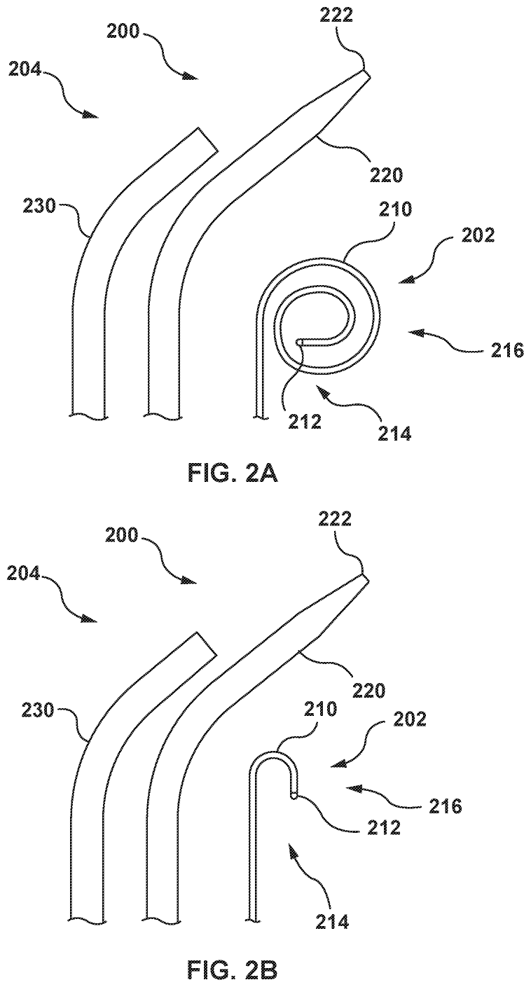

FIGS. 2A and 2B are an illustration of a catheter assembly 200 that incorporates embodiments of devices that may be utilized during the course of the procedure as described further hereinbelow. Assembly 200 is used for puncturing tissue such as for creating a transseptal puncture through a septum of a heart. The assembly 200 comprises a tissue puncturing device 202, and supporting catheters 204. In this embodiment, supporting catheters 204 comprises a sheath 230 and a dilator 220 that are selectively usable with the tissue puncture device 202. Providing a separate puncture device 202 and supporting catheters 204 enhance procedural efficiency by facilitating exchange and positioning. In some embodiments, the tissue puncturing device 202 is substantially flexible. In some such examples, the flexible puncture device 202 comprises an energy delivery device 214 that is configured to deliver energy in order to puncture tissue. In other embodiments, the tissue puncturing device is substantially flexible and comprises a sharp pointed distal tip end section (not shown in the figure). In some such embodiments, the end section may be curved and atraumatic.

In specific examples of the distal portions of assembly 200, as shown in FIGS. 2A and 2B, the puncture device 202 comprises a substantially atraumatic distal tip 212 wherein the puncture device 202 is substantially atraumatic. In some embodiments, the puncture device 202 comprises an energy based puncture device 214 such as a substantially flexible energy based puncture device that has an energy delivery portion or component at the distal tip 212 for delivering energy in order to puncture tissue. In a specific instance of this example, the puncture device 202 comprises a flexible (radiofrequency) RF wire 210 that has a distal electrode tip 212 for delivering radiofrequency in order to puncture tissue. In some instances, the RF wire 210 is a flexible wire which is generally electrically insulated save for selected distal regions such as the distal electrode tip 212.

In some embodiments of the assembly 200, as shown in FIGS. 2A and 2B, the supporting catheters comprise a dilator 220 and a sheath 230. Dilator 220 and sheath 230 each defines a respective lumen through which devices may be inserted.

In some embodiments, the sheath 230 is a steerable sheath. In some embodiments, the steerable sheath is unidirectional, i.e. it allows deflection in a single direction. In other embodiments, a bi-directional sheath may be used. Alternately, in some embodiments, a fixed curve sheath may be utilized in place of an articulating sheath, depending on the tortuosity of the vasculature.

In some examples, the dilator 220 provides stiffness to the assembly 200 to facilitate force to be transmitted to a distal end of the assembly 200. In other examples, the sheath 230 is usable with the dilator 220 to provide stiffness to the assembly 200 to enable force or torque to be transmitted to a distal end of the assembly 200. In some such examples, the sheath 230 may be coupled to the dilator 220 which enables force and/or torque transmission using one or more of the components (i.e., the sheath 230 or the dilator 220). In other words, the user may not have to manipulate the sheath 230 and the dilator 220 (the user may just manipulate the sheath 230 or the dilator 220) and the RF wire 210 follows the guidance and/or direction of the sheath 230 and/or the dilator 220.

In some examples of the embodiments of FIGS. 2A and 2B, a flexible dilator 220 is used with a steerable sheath 230 to access a region of tissue within a patient's body. The steerable sheath 230 has a range of deflection angles and can achieve a range of curvature upon actuation. The dilator includes a substantially flexible or soft section (not shown) that provides minimal resistance to deflection and is operable to be deflected under guidance to allow the dilator 220 to reach a desired site within a region of tissue within the patient's body to facilitate advancement of the distal end region. The flexible region allows the dilator 220 to conform to the curvature of the steerable sheath 230 that is achieved through actuation of the steerable sheath. In some embodiments, the distal end of the dilator 220 extends beyond the distal end of the sheath 230 to improve access to the target tissue within the patient's body.

In a specific embodiment, an 8.5 French steerable sheath 230 with a 45 cm usable length and an 8.5 French dilator 220 with a usable length of 67 cm is used. The dilator 220 tapers down to an outer diameter (OD) of about 0.046″ (about 1.2 mm) and an inner diameter (ID) of about 0.036″ (about 0.9 mm) at the distal tip 222.

In other embodiments, the supporting catheter is a hybrid dilator. The hybrid dilator comprises a lumen for receiving the puncturing device, a dilator shaft being structured to provide support for the puncturing device, and a distal tip having an outer diameter which tapers down to an outer diameter of the puncturing device. The hybrid dilator provides a dual functionality of a sheath and a dilator for facilitating a transseptal puncture procedure.

In accordance with embodiments of the present invention, as described herinabove, FIGS. 2A and 2B illustrate embodiments of a medical device operable to be guided to a tissue site to puncture tissue and to function as a rail for installing devices thereupon. Such embodiments provide efficiencies to medical procedures in which they are utilized as they perform multiple functions and thereby reduce the amount of device exchanges that need to be performed. The “hybrid” medical devices further facilitate the access and puncture of a tissue site upon insertion at a particular access site on a patient's body.

FIGS. 2A and 2B further illustrate embodiments of a substantially flexible energy based puncturing device such as an RF wire 210 that is sufficiently flexible to enable access to heart tissue, such as a septum, from, for example, a transhepatic approach. An active tip 212 at the distal end is operable to deliver energy for puncturing tissue such as a heart septum to create a puncture site through which the RF wire 210 can be advanced, for example to enter the left atrium. In a specific example the RF wire 210 has an outer diameter (OD) of 0.035″ and a wire length of 180 cm. In another example. The RF wire 210 has an outer diameter (OD) of 0.032″. In a further example, the RF wire 210 has a radiopaque marker.

FIG. 2A further shows a specific embodiment of a pigtail RF wire 210 where the distal section 216 is biased to form a coil for anchoring the RF wire 210 beyond the puncture site. FIG. 2B shows a specific embodiment of a J-tip RF wire 210 where the distal section 216 substantially forms a “J”. Typically, when distal section 216 is advanced out of a dilator and beyond the septum, the biased curves act as an anchor in the left atrium, providing a rail into the left atrium.

### Methods

Access for a transseptal puncture may be provided through vasculature using a guidewire. A sheath and dilator assembly may then be advanced over the guidewire, to reach the target site. Access to the vasculature for a transseptal puncture is traditionally achieved through the femoral vein.

In some such applications, the femoral vein or other conventional access points (for example the jugular vein) may not be practical. For example, in patients with an azygous continuation of the IVC, it may not be possible to gain access to the right atrium using a femoral approach. Occluded or stenosed vasculature may also prevent the use of traditional access sites. In other such applications, traditional access sites (such as femoral venous access or jugular vein access) are used to provide access to auxiliary devices for the procedure.

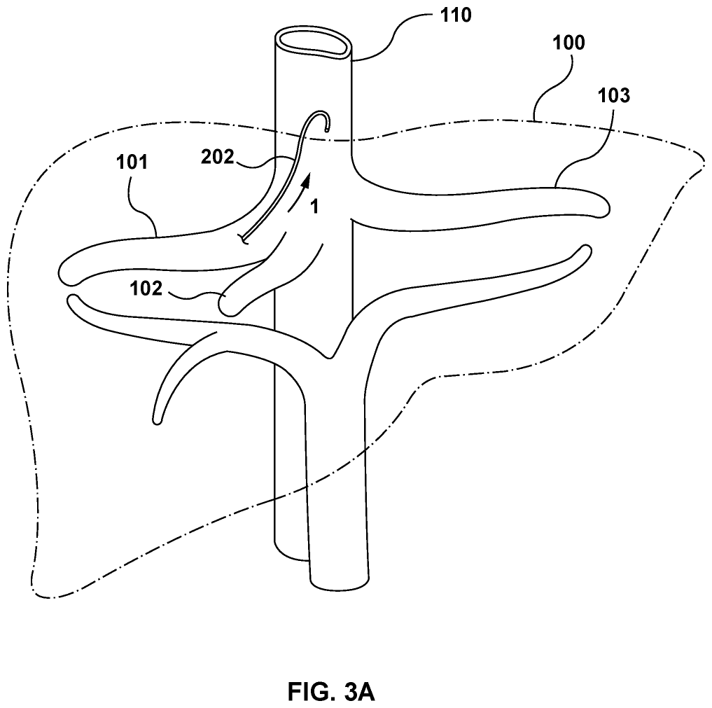

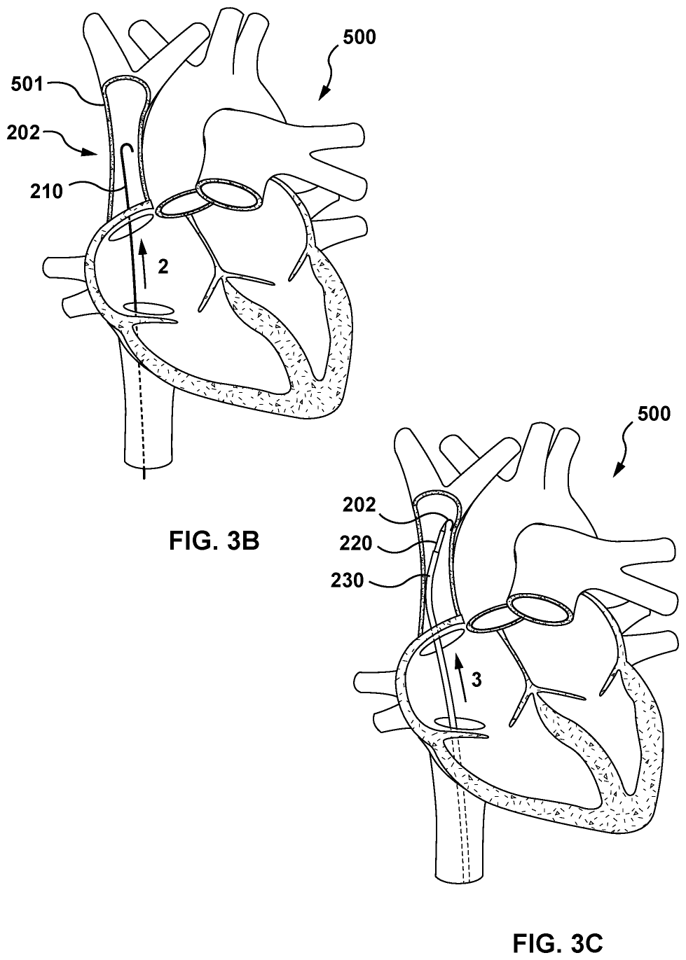

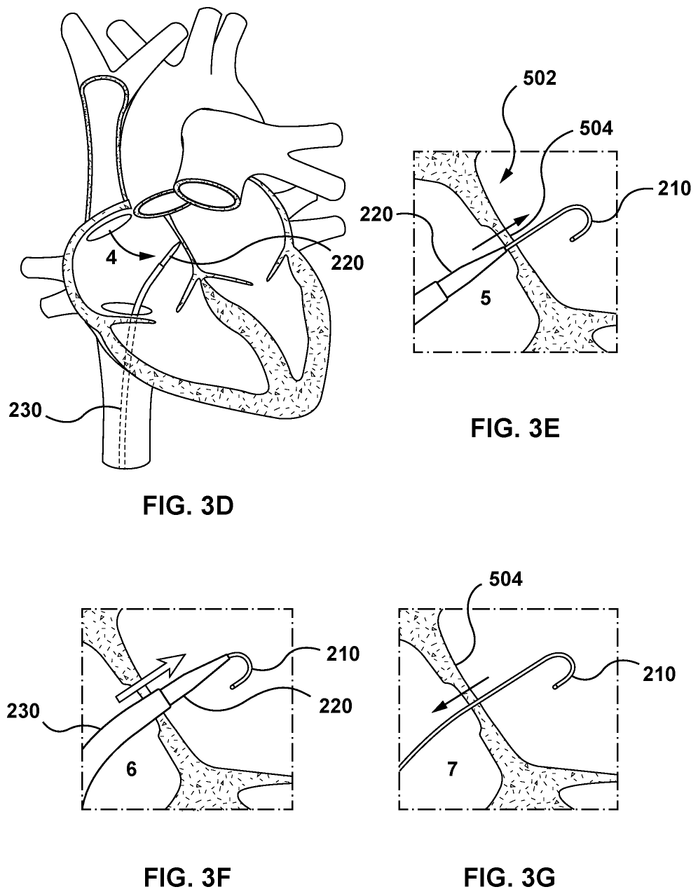

In some embodiments of the present invention, with reference now to FIGS. 3A-3G, a method is disclosed for puncturing tissue. In step [1] (shown in FIG. 3A) the inferior vena cava (IVC) is accessed through a hepatic vein 101, 102 or 103. In some embodiments, prior to accessing the hepatic vein 101, 102 or 103, the hepatic veins and the areas are visualized or mapped using visualization or mapping techniques. This visualization or mapping allows the physician to select an access point (i.e., a particular hepatic vein) which provides a clear, unobstructed path to the target tissue. In some embodiments, the physician may select a lower hepatic vein (not shown) as an access point. In some embodiments, the hepatic vein is accessed using an access device such as a Tuohy needle (not shown). Because puncturing device 202 is flexible and atraumatic, it is capable of navigating vasculature through the hepatic vein 101 to the IVC 110 without damaging any surrounding structures, and without the need of an exchange or guidewire.

Once the hepatic vein 101 is reached, step [2] involves accessing a region of tissue within a patient's body by advancing a device (such as puncture device 202) towards said region, as shown in FIG. 3B. In this embodiment, the flexible puncturing device 202 is advanced through the IVC 110 and into the superior vena cava (SVC) 501 adjacent a heart 500.

In step [3] (shown in FIG. 3C), the dilator 220 and/or sheath 230 are tracked over the puncture device 202. The dilator 220 and/or sheath 230 support the puncture device as shown in FIG. 3C by providing rigidity and stiffness. In step [4], the combined apparatus is dropped down from the superior vena cava (SVC) into the right atrium of heart 500. From there, the combined apparatus is manually manipulated towards a fossa ovalis. Visualization techniques such as fluoroscopy, intracardiac electrocardiography, or other means may be used to assist in locating the distal tip of the puncture device 202 at an appropriate position on the fossa ovalis 504 (FIG. 3E) along a septum 502 of the heart 500 (shown in FIG. 3D). In addition, sheath 230 may be steerable such that the positioning of the distal tip of the puncture device 202 may be further adjusted or fine-tuned. The steerable sheath may also facilitate navigating through tortuous anatomy. The steerable sheath allows for position and/or guidance of the flexible puncture device to locate and position on the fossa ovalis 504.

In embodiments of the present invention, as shown in FIG. 3E, the method additionally comprises: [5] a step of puncturing through the target tissue site using a puncturing device (such as the RF wire 210) after the positioning step (step [4]). The dilator 220 and/or sheath 230 support the puncturing device (such as RF wire 210) during the puncturing and the system allows for energy to be delivered from the puncture device 202 (such as RF wire 210) to puncture through the septum to access the left atrium. In such embodiments, the steps of accessing [2] and puncturing [5] are performed using the same puncturing device.

In some embodiments of the present invention, the step [5] of puncturing through the target tissue site comprises the step of puncturing through the fossa 504 to gain access to a left side of the heart 500. This enables one or more devices of the assembly 200, such as the dilator 220 and/or the sheath 230 of the assembly 200 to be tracked over the puncturing device 202 such as the RF wire 210 into the left side of the heart.

The method may additionally comprise a step of anchoring, as shown in FIG. 3E. In an embodiment, anchoring is performed using the puncture device (such as RF wire 210) after the step [5] of puncturing through the target tissue site. In this embodiment, anchoring is enabled by the curvature of puncturing device, RF wire 210 after it has punctured the septum. FIG. 3E illustrates that once the RF wire 210 is no longer supported by the sheath 230 or dilator 220, the distal portion 216 of the RF wire forms a J-tip. The J-tip of the puncturing device 202 (such as RF wire 210) allows the puncturing device to remain on the other side of the target tissue site. Anchoring allows access through the target tissue site to the other side of the target tissue site to be maintained.

By maintaining access to the other side of the target tissue, one or more devices can be tracked over the puncture device to the other side of the target tissue site. This is illustrated in FIG. 3F. In this embodiment, the steps of accessing, puncturing and anchoring are performed using the same device. In one embodiment, the one or more devices may include a sheath 230 and/or dilator 220. Tracking a sheath 230 and/or dilator 220 allows the hole created by the puncturing device to be enlarged. This is desirable in some cases where additional devices with larger diameters may be needed for visualization or treatment.

The puncture device such as the RF wire 210 may be left to maintain access to the left side of the heart, as shown in FIG. 3G. The dilator 220 and/or the sheath 230 may be removed or retracted to allow anchoring using the RF wire 210. The RF wire 210 thus functions as a rail to guide one or more auxiliary devices (such as visualization or treatment devices) to the left side of the heart. In some such examples, the RF wire 210 provides a substantially stiff rail to guide the one or more devices to the left side of the heart while being substantially atraumatic to minimize damage to the tissue.

In some such embodiments of the present invention, the step of anchoring to maintain access through the target tissue site comprises advancing the device (such as the puncture device, RF wire 210) through the fossa to the left side of the heart to maintain access to the left side of the heart. The step additionally comprises a step of removing the dilator 220 and/or sheath 230 and leaving the puncture device such as RF wire 210 to maintain access to the region of tissue such as the left side of the heart. As such, the step of anchoring comprises removing the dilator 220 and/or sheath 230 to enable anchoring by allowing the RF wire 210 to remain positioned and maintain access to the left side of the heart. In some such embodiments of the present invention, the steps of accessing, puncturing and anchoring are performed using a flexible energy based puncture device (such as RF wire 210). The use of the RF wire 210 as a guidewire, puncture device, anchor, and/or exchange wire results in a reduced number of devices needed, and a reduced workflow. The benefit of minimizing exchanges, in addition to reducing time/steps, is minimizing risk of infection and reduces unnecessary exchanges which may lead to increased risk of embolism, strokes, etc.

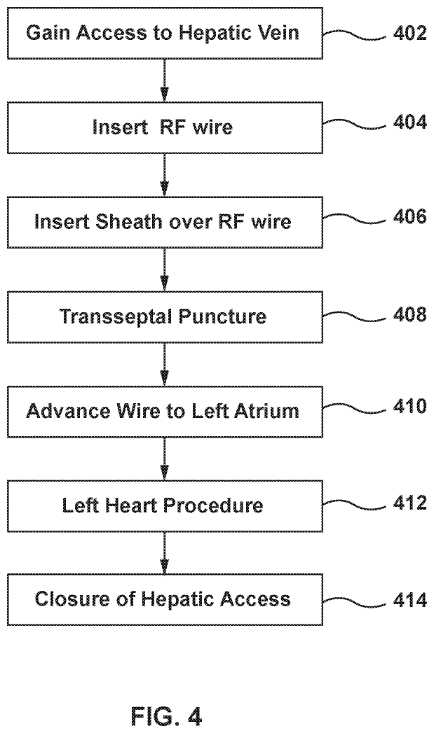

In accordance with embodiments of the present invention, an exemplary method is provided for performing a transseptal procedure using a hepatic access (see FIG. 4). The method comprises the following steps: at step 402, access is gained to hepatic vein. In a specific embodiment, the right hepatic vein is accessed. In other embodiments, the middle or left hepatic vein is accessed. In some other embodiments, a lower hepatic vein is accessed. In some embodiments, the hepatic vein is accessed using an access device (such as a Tuohy or Chiba needle). In a specific embodiment, a 17 gauge Tuohy needle is used to access the hepatic vein.

At step 404, the puncture device 202 such as an RF wire 210 is inserted into the hepatic vein. RF wire 210 is sufficiently flexible to be tracked up the IVC and is placed in the SVC or right atrium. In a specific embodiment, the RF wire 210 has an outer diameter (OD) of 0.032″.

After the RF wire 210 has been inserted and is positioned near the target area, the method continues to step 406, insert sheath over RF wire. In some embodiments, the sheath 230 may comprise a fixed angle. In other alternatives, sheath 230 may be steerable. In some such examples, the sheath is steerable to allow for in-situ adjustment of the sheath, for example an angle of the sheath, to enable it to aim at a selected position or location along the septum. In a specific embodiment the sheath 230 is an 8.5 French steerable sheath.

The use of a steerable sheath in combination with an RF wire, which is flexible, provides advantages relative to conventional needles when advancing the steerable sheath from the hepatic vein to the right atrium. In particular, a conventional needle typically includes a fixed angle or curve and may only be torqued, which may severely limit the options available to the physician for aiming or guiding the needle to the septum. A conventional needle is not ideal for use with a steerable sheath as the rigidity of the conventional needle limits the extent to which it may be steered. An RF wire, on the other hand, is much more flexible and is capable of taking on the shape of the steerable sheath with less effort.

In some embodiments, step 406 comprises tracking a sheath 230 and/or dilator 220 assembly over the RF wire 201. In such embodiments, the dilator 220 is tracked over the RF wire 210 and the sheath 230 is tracked over the dilator 220. In some embodiments, the sheath 230 is a steerable sheath and the dilator 220 is sufficiently flexible to be movable by the sheath such that it is readily conformable to the shape of the steerable sheath 230. Thus, the shape of the combined apparatus of sheath 230, dilator 220, and RF wire 210 may be adjusted through actuation of the steerable sheath. In a specific example, the sheath 230 is an 8.5 French steerable sheath and the dilator 220 is an 8.5 French dilator. In this specific example, the outer diameter of the dilator corresponds with the inner diameter of the steerable sheath. The combined apparatus is sufficiently stiff to tent the fossa ovalis.

At step 408, transseptal puncture, the puncture device such as an RF wire 210 is configured to puncture the septum. Catheter assembly 200 is positioned at the fossa ovalis of the septum of the heart. The catheter assembly creates tenting of the septum to provide contact with the target tissue. Once positioned at the target tissue, an active tip 212 at the distal end of the RF wire 210 is operated to deliver energy to puncturing the tissue and creating a puncture site in the septum.

In step 410, the RF wire is advanced to the left atrium. The puncture device 202 such as the RF wire 210, advances through the hole created by the puncture into the left atrium. In some embodiments, the method further comprises anchoring the RF wire 210 in the left atrium. This may be achieved by providing the RF wire 210 with a distal portion that has a pigtail or spiral shape (FIG. 2A), a J-tip (FIG. 2B), or some other suitable shape. The pigtail spiral and J-tip provide anchoring support for the RF wire 210 as the shape at the distal portion of the RF wire 210 makes it harder for the wire 210 to fall out of the hole created by the puncture. Additionally, because the distal portion of the RF wire 210 is atraumatic, it may also be lodged in a vein or other cavity in the left atrium to further secure it.

Once anchored in the left atrium, the RF wire 210 can be used as an exchange wire, providing a track for devices requiring access to the left atrium for a left heart procedure. In some embodiments, sheath 230 and/or dilator 220, are advanced to the left atrium over the RF wire 210 to enlarge the hole or provide a mechanism for delivering other devices. In some embodiments, the septum is dilated by a dilator. The tapered distal end of the dilator dilates or increases the size of the puncture site. In other embodiments, a balloon dilator is used (not shown). The balloon dilator comprises a dilation balloon operable to be in a collapsed stated or expanded state. The dilation balloon is positioned within the puncture site in a collapsed state, and is inflated to an expanded state to dilate or increase the size of the puncture site. The dilatation balloon can be any type of dilatation balloons (that are known in the art) used to increase the size of the puncture site in transseptal crossing. In a further example, dilatation balloon can be attached to the dilator 220 shaft (not shown) and can be fabricated from materials known in the prior art (for example, PET, polyamide, cross-linked polyolefins, or the like).

In some embodiments, the sheath 230 is used in the left heart procedure and the RF wire 210 is retracted and the devices for the left heart procedure are advanced through the sheath 230. Left heart procedures include left atrial appendage closure, mitral valve repair, pulmonary vein ablation, etc. In a specific example, the sheath 230 is a steerable sheath that is used for the left heart procedure.

In other embodiments, the sheath 230 and/or dilator 220 are not used for the left heart procedure. In such embodiments, the RF wire 210 is anchored in the left atrium and the sheath 230 and/or dilator 220 are retracted and the left heart procedure devices are advanced over the RF wire 210 across the septum into the left side of the heart.

The left heart procedure 412 is performed. The access point of the hepatic vein is in closer proximity to the heart compared to the femoral access point. This allows larger catheters and devices to be used. The shorter distance between the access point and the heart provides greater control of the devices. Access through the hepatic vein enables physicians to use tools designed specifically to approach the heart from the inferior vena cava. This allows physicians to use practiced techniques and tools for the left heart procedure. In some specific examples, for procedures such as a left atrial appendage occlusion procedure, an approach from the inferior vena cava is more direct.

After the left heart procedure of step 412 is complete, the devices are removed and the hepatic access is closed 414. In some embodiments, a gel foam is used to close the hepatic access. In some other embodiments, a closure device, such as a coil or plug, is used to close the hepatic access.

### Further Examples

1. A method for puncturing tissue a target tissue site within a patient, the method comprising the steps of:

(a) accessing a hepatic vein within the patient using an access device;

(b) inserting a flexible puncture device into the hepatic vein;

(c) advancing the flexible puncture device from the hepatic vein to an inferior vena cava of the patient;

(d) advancing the flexible puncture device through the inferior vena cava to a heart of the patient;

(e) advancing a supporting catheter comprising a lumen over the flexible puncture device, whereby the flexible puncture device is within the lumen and supported by the supporting catheter;

(f) positioning a distal tip of the flexible puncture device and supporting catheter against the target tissue site and creating a puncture in said tissue; and

(g) advancing the flexible puncture device through the target tissue.

2. The method of example 1, wherein the supporting catheter supports the flexible puncture device to enable force and torque to be transmitted to a distal end of the supporting catheter and puncture device.
3. The method of any one of examples 1 or 2, wherein the flexible puncture device is energy based.
4. The method of example 3, wherein the flexible puncture device is a radiofrequency wire.
5. The method of any one of examples 1 to 4, wherein the flexible puncture device is a pigtail wire.
6. The method of any one of examples 1 to 4, wherein the flexible puncture device is a J-tip wire.
7. The method of any one of examples 1 to 6, wherein the flexible puncture device is sufficiently flexible to be tracked through vasculature of the patient.
8. The method of any one of examples 1 to 7, wherein the supporting catheter is a steerable sheath.
9. The method of any one of examples 1 to 8, wherein after step (g), the method further comprising,

(h) anchoring the flexible puncture device after puncturing through the target tissue site, to maintain access through the target tissue site to the other side of the target tissue site, in order to allow one or more additional devices to be tracked over the flexible puncture device to the other side of the target tissue site.

10. The method of example 9, where after step (h), the method further comprising,

(i) performing a left heart procedure.

11. The method of example 10, wherein after step (i), the method further comprising,

(j) removing the devices and closing the hepatic access.

12. The method of any one of examples 1 to 11, wherein the target tissue site is a septum of the heart.

13. The method of example 12, wherein step (f) comprises dropping the device down from a superior vena cava into the heart of the patient to locate a fossa along the septum of the heart to position the flexible puncture device at the fossa.
14. The method of any one of examples 8 to 13, wherein the step of positioning the device at the target tissue site comprises actuating the steerable sheath to position the flexible puncture device at a fossa along a septum of the heart.
15. The method of any one of examples 1 to 14, wherein the supporting catheter comprises a dilator and a sheath, wherein step (c) comprises inserting the dilator over the flexible puncture device and inserting the sheath over the dilator.
16. The method of example 15, wherein the sheath is a steerable sheath, and the dilator is substantially flexible to conform to the curve of the steerable sheath that is achieved through actuation of the steerable sheath.
17. The method of example 15, wherein after step (g), the method further comprising,

(h) advancing the dilator through the puncture in the tissue over the flexible puncture device.

18. The method of example 17 wherein after step (h), the method further comprising,

(i) advancing the sheath through the puncture in the tissue over the dilator.

19. The method of any one of examples 1 to 18, wherein after step (g), the method further comprising

(h) advancing the sheath through the puncture in the tissue over the flexible puncture device.

20. The method of any one of examples 1 to 19, wherein the hepatic vein is a right hepatic vein.
21. The method of any one of examples 1 to 19, wherein the hepatic vein is a middle or left hepatic vein.
22. The method of any one of examples 1 to 19, wherein the hepatic vein is a lower hepatic vein.
23. The method of any one of examples 1 to 19, wherein the access device is a Tuohy needle.
24. The method of any one of examples 1 to 19, wherein prior to step a), the method further comprises visualizing hepatic veins in the patient and selecting a hepatic vein.
25. The method of any one of examples 1 to 19, wherein the flexible puncture device comprises a lumen and one or more apertures.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended examples. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The embodiments of the invention described above are intended to be exemplary only. Thus, it is intended that the present invention include these variations and modifications, provided they come within the scope of the appended claims and their equivalents.

We claim:

1. A method for puncturing tissue a target tissue site within a patient, the method comprising the steps of:
(a) accessing a hepatic vein within the patient using an access device;
(b) inserting a flexible puncture device into the hepatic vein;
(c) advancing the flexible puncture device from the hepatic vein to an inferior vena cava of the patient;
(d) advancing the flexible puncture device through the inferior vena cava to a heart of the patient;
(e) advancing a supporting catheter comprising a lumen over the flexible puncture device, whereby the flexible puncture device is within the lumen and supported by the supporting catheter;
(f) positioning a distal tip of the flexible puncture device and supporting catheter against the target tissue site and creating a puncture in said tissue; and
(g) advancing the flexible puncture device through the target tissue.

2. The method of claim 1, wherein the supporting catheter supports the flexible puncture device to enable force and torque to be transmitted to a distal end of the supporting catheter and puncture device.

3. The method of claim 2, wherein the flexible puncture device is energy based.

4. The method of claim 3, wherein the flexible puncture device is a radiofrequency wire.

5. The method of claim 4, wherein the radiofrequency wire is a pigtail wire.

6. The method of claim 4, wherein the radiofrequency wire is a J-tip wire.

7. The method of claim 1, wherein the flexible puncture device is sufficiently flexible to be tracked through vasculature of the patient.

8. The method of claim 1, wherein the supporting catheter is a steerable sheath.

9. The method of claim 1, wherein after step (g), the method further comprising,
(h) anchoring the flexible puncture device after puncturing through the target tissue site, to maintain access through the target tissue site to the other side of the target tissue site, in order to allow one or more additional devices to be tracked over the flexible puncture device to the other side of the target tissue site.

10. The method of claim 9, where after step (h), the method further comprising,
(i) performing a left heart procedure.

11. The method of claim 10, wherein after step (i), the method further comprising,
(j) removing the devices and closing the hepatic access.

12. The method of claim 1, wherein the target tissue site is a septum of the heart.

13. The method of claim 12, wherein step (f) comprises dropping the device down from a superior vena cava into the heart of the patient to locate a fossa along the septum of the heart to position the flexible puncture device at the fossa.

14. The method of claim 8, wherein the step of positioning the device at the target tissue site comprises actuating the steerable sheath to position the flexible puncture device at a fossa along a septum of the heart.

15. The method of claim 1, wherein the supporting catheter comprises a dilator and a sheath, wherein step (c) comprises inserting the dilator over the flexible puncture device and inserting the sheath over the dilator.

16. The method of claim 15, wherein the sheath is a steerable sheath, and the dilator is substantially flexible to conform to the curve of the steerable sheath that is achieved through actuation of the steerable sheath.

17. The method of claim 15, wherein after step (g), the method further comprising,
(h) advancing the dilator through the puncture in the tissue over the flexible puncture device.

18. The method of claim 17 wherein after step (h), the method further comprising,
(i) advancing the sheath through the puncture in the tissue over the dilator.

19. The method of claim 1, wherein after step (g), the method further comprising
(h) advancing the sheath through the puncture in the tissue over the flexible puncture device.

20. The method of claim 1, wherein prior to step a), the method further comprises visualizing hepatic veins in the patient and selecting a hepatic vein.

21. The method of claim 1, wherein the flexible puncture device comprises a lumen and one or more apertures.
